(19) United States
(12) Patent Application Publication
(10) Pub. No.: US 2004/0087572 A1
(43) Pub. Date: May 6, 2004

(54) Method for inhibiting cataracts

(76) Inventors: Pamela Wang Anderson, Indianapolis, IN (US); Andreas Sashegyi, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/380,450

(22) PCT Filed: Oct. 12, 2001
(86) PCT No.: PCT/US01/27766

(51) Int. Cl. A61K 31/55; A61K 31/453; A61K 31/4025

(52) U.S. Cl. 514/217.03; 514/324; 514/422

(57) ABSTRACT

This invention relates to a method for inhibiting cataracts comprising administering to a patient in need thereof an effective amount of a compound of the formula (I); or a pharmaceutical salt or solvate thereof wherein: R1 and R3 are independently hydrogen, methyl, benzoyl, substituted benzoyl, or C(O)—(C1-C6 alkyl); R2 is selected from the group pyrolidin-1-yl, piperidin-1-yl, and hexamethyleneimin-1-yl; where the R2 group is optionally the N-oxide.

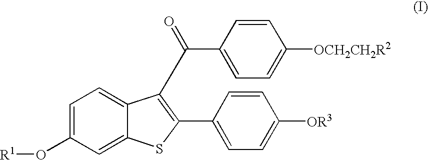

The term “cataract” is a general term for any pathological condition in which the normal transparency of the ocular lens is substantially diminished. More than one million cataract extractions are performed annually in the United States, and it is estimated that 5 to 10 million individuals become visually disabled each year due to cataracts.

Although often regarded as an inevitable accompaniment of advancing age, cataracts may develop at any time in life, even before birth. Risk factors for cataract formation include metabolic disorders (e.g., diabetes), exposure to toxic agents in the environment (e.g., ultraviolet radiation, ionizing radiation), drug side effects, and inherited traits. Clinical experience suggests that the natural course of different types of cataracts are distinct. However, objective, quantitative data is generally lacking.

Development of anti-cataract agents has been hampered, in part, by the lack of a good animal model of human cataract. Consequently, putative anti-cataract agents may be evaluated for efficacy in a variety of different models which, to the extent that they are understood at all, are thought to occur by different mechanisms. For example, radiation-induced cataract is generally believed to result from oxidative damage to the lens. Diabetic cataract is thought to be due to the accumulation of polyols (such as sorbitol) in the lens, resulting from increased activity of the enzyme aldose reductase. According to The Royal College of Surgeons (RCS), hereditary cataract is thought to be due to the action of products released by the retina.

In contrast to the understanding of cataract pathogenesis, the cellular structure of the lens is fairly well characterized. The lens exhibits a high degree of regularity, consisting of fiber cells with hexagonal cross sections packed together to create a very regular parallel array of fiber cells which stretch from anterior to posterior pole. The lens fiber cells lose all intracellular organelles that could contribute to light scattering during the process of differentiation and the cytoplasmic protein concentration increases markedly.

Approximately 35% to 60% of the total mass of the lens consists of structural proteins, the remainder being water. More than 90% of the total lens protein consists of alpha, beta, and gamma crystallins, a group of structural proteins found at extremely high concentrations (in excess of 300 mg/ml) in the lens cell cytoplasm. The cytoplasmic concentration of the crystallins throughout the lens occurs along a continuous radial concentration gradient, in which the concentration is greatest in cells at the nucleus and decreases in peripheral cells of the lens cortex. The crystallin distribution determines the mean index of refraction and index gradient, which are in turn responsible for the special optical properties of the animal lens.

An important optical property is lens transparency. In the normal lens, incident light is scattered in all directions by the macromolecular constituents of the lens. If the individual wavelets of the scattered light interfere destructively with one another, the lens is transparent. Destructive interference takes place in the normal lens because of the existence of short range order in the relative positions of the crystallins. If the uniformity of the protein concentration is sufficiently perturbed, a substantial fraction of the incident light is scattered in directions away from the forward direction. The scattering results in a distortion of the wave front of the transmitted light, and in opacity of the lens tissue. The opacity is responsible for visual impairment in cataract diseases.

Cataracts are the leading cause of blindness in humans worldwide, and surgery remains the primary form of treatment. Cataracts in animals also pose a significant veterinary problem. To date, a compound for in vivo administration to humans or other animals has not been demonstrated to prevent cataracts of diverse origin. Further, in vivo reversal of the initiation of cataract formation has not been successfully demonstrated.

Therefore, there is a significant need for an effective nonsurgical method for treating or preventing cataractogenesis in humans and other animals. This method should utilize compounds which are relatively safe and which may be conveniently administered. The present invention fulfills these needs and provides other related advantages.

This invention relates to a method for inhibiting cataracts comprising administering to a patient in need thereof an effective amount of a compound of the formula

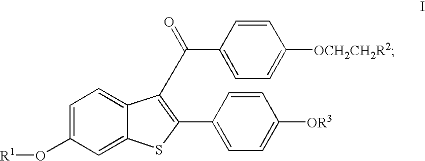

or a pharmaceutical salt or solvate thereof wherein:

R¹ and R³ are independently hydrogen, methyl, benzoyl, substituted benzoyl, or C(O)—(C1-C6 alkyl);

R² is selected from the group pyrolidin-1-yl, piperidin-1-yl, and hexamethyleneimin-1-yl; where the R² group is optionally the N-oxide.

As used herein, the term “inhibit” is defined to include its generally accepted meaning which includes preventing, prohibiting, restraining, and slowing, stopping or reversing progression, or severity, and holding in check and/or treating existing characteristics. The present method includes both medical therapeutic and/or prophylactic treatment, as appropriate.

The term “estrogen deficient” refers to a condition, either naturally occurring or clinically induced, where a woman can not produce sufficient estrogenic hormones to maintain estrogen dependent functions, e.g., menses, homeostasis of bone mass, neuronal function, cardiovascular condition, etc. Such estrogen deficient situations arise from, but are not limited to, menopause and surgical or chemical ovarectomy, including its functional equivalent, e.g., medication with GnRH agonists or antagonists, ICI 182780, and the like.

The term “patient” refers to a warm-blooded animal or mammal. It is understood that guinea pigs, dogs, cats, rats, mice, hamsters, rabbits and primates, including humans, are examples of patients within the scope of the meaning of the term.

General terms used in the description of compounds herein described bear their usual meanings. For example, “C₁-C₆ alkyl” refers to straight or branched aliphatic chains of 1 to 6 carbon atoms including methyl, ethyl, propyl, iso-propyl, n-butyl, pentyl, hexyl and the like.

The term “substituted phenyl” refers to a phenyl group alone or having one or more substituents selected from the group consisting of C₁-C₄ alkyl, C₁-C₄ alkoxy, hydroxy, nitro, chloro, fluoro, or tri(chloro or fluoro)methyl. “OC₁-C₄ alkyl” refers a C₁-C₄ alkyl group attached through an oxygen bridge such as, methoxy, ethoxy, n-propoxy, iso-propoxy, and the like.

The term “substituted benzoyl” refers to benzoyl group having one to five substituents selected independently from the group: C₁-C₄ alkyl, C₁-C₄ alkoxy, hydroxy, nitro, chloro, fluoro, or tri(chloro or fluoro)methyl.

The term “pharmaceutical salt” refers to either acid or base addition salts which are known to be non-toxic and are commonly used in the pharmaceutical literature. Commonly used acid addition salts are inorganic salts formed by the addition of sulfuric acid, nitric acid, hydrochloric acid, hydrobromic acid phosphoric acid, phosphorous acid and the like; or organic salts formed by the addition of acetic acid, formic acid, benzoic acid, citric acid, methanesulfonic acid and the like. Commonly used basic addition salts are the salts formed by alkali or alkaline earth hydroxides, ammonium hydroxide, alkyl or aromatic amines and the like. A preferred salt of this invention is the hydrochloride salt.

The term “solvate” refers to a molecular complex of a compound of formula I with one or more solvent molecules. Such solvent molecules would be those commonly used in the pharmaceutical literature, which are known to be innocuous to the recipient, e.g., water, ethanol, and the like.

The compounds of this invention are derivatives of centrally located carbon, i.e., the “—CO—” moiety in formula I, thus derivatives are methanones, e.g., a compound of ACO-B, would be named [A] [B]methanone. Further the compounds of formula I are derivatives of benzo[b]thiophene which is named and numbered according to the Ring Index, The American Chemical Society, as follows:

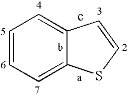

Thus, raloxifene hydrochloride, which is a preferred embodiment of this invention, is a compound of formula I, where R¹ and R³ are both hydrogen and R is a piperidinyl ring, the hydrochloride salt thereof. Raloxifene hydrochloride is named [2-(4-hydroxyphenyl)-6-hydroxybenzo[b]thie-3-yl][4-[2-(1-piperidenyl)ethoxy]phenyl]methanone hydrochloride. It is sold commercially as EVISTA® and is indicated in the United States for the prevention and treatment of osteoporosis.

The compounds of Formula I and salts and solvates thereof may be prepared according to known procedures, such as those detailed in U.S. Pat. Nos. 4,133,814, 4,418,068, 5,631,369, 5,731,327, 5,731,342, 5,750,688 and 5,977,383, each of which is incorporated by reference herein as if fully set forth. Preferred crystalline forms, particle sizes and pharmaceutical formulations are disclosed in U.S. Pat. Nos. 5,641,790, 5,731,327, 5,747,510, and 5,811,120, each of which is incorporated by reference herein as if fully set forth.

The compounds of formula I are members of a group of compounds previously known as antiestrogens, but which have selective estrogenic agonist and antagonist pharmacologic activities. For example, formula I compounds act as estrogen agonists in treating pathologic sequelae caused by the cessation of menses in females (see: Draper et al., “Effects of Raloxifene (LY139481 HC1) on Biochemical Markers of Bone and Lipid Metabolism in Healthy Postmenopausal Women”, Hong Kong, Fourth Int'l. Symp. on Osteoporosis, Mar. 29, 1993; U.S. Pat. Nos. 5,393,763, 5,464,845, and 5,391,557).

Pharmaceutical formulations can be prepared by procedures known in the art, such as, for example, in European Published Application 670162A1, published Sep. 6, 1995, and in WO 97/35571 published Oct. 2, 1997, both of which are herein incorporated by reference. For example, a compound of formula I can be formulated with common excipients, diluents, or carriers, and formed into tablets, capsules, and the like.

Examples of excipients, diluents, and carriers that are suitable for formulation include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonire; and lubricants such as talc, calcium and magnesium stearate and solid polyethyl glycols. Final pharmaceutical forms may be: pills, tablets, powders, lozenges, syrups, aerosols, saches, cachets, elixirs, suspensions, emulsions, ointments, suppositories, sterile injectable solutions, or sterile packaged powders, depending on the type of excipient used.

Additionally, raloxifene and its pharmaceutically acceptable salts are suited to formulation as sustained release dosage forms. The formulations can also be so constituted that they release the active ingredient only or preferably in a particular part of the intestinal tract, possibly over a period of time. Such formulations would involve coatings, envelopes, or protective matrices which may be made from polymeric substances or waxes.

As used herein, the term “effective amount” means an amount of compound of the present invention which is capable of inhibiting cataracts in a patient, preferably a human, and most preferably a post-menopausal woman.

The particular dosage of raloxifene or a pharmaceutically acceptable salt thereof required to constitute an effective amount according to this invention will depend upon the particular circumstances of the conditions to be treated. Considerations such as dosage, route of administration, and frequency of dosing are best decided by the attending physician. Generally, accepted and effective dose ranges for oral or parenteral administration will be from 10 mg to 800 mg, and more typically between 20 mg and 100 mg. Furthermore, an effective minimum dose for oral or parenteral administration of raloxifene or a pharmaceutically acceptable salt thereof is about 1, 5, 10, 15, or 20 mg. Typically, an effective maximum dose is about 800, 120, 60, 50, or 40 mg. A typical dosage range may be any combination of the above specific minimum and maximum doses. A particularly effective amount is 60 mg of raloxifene hydrochloride (56 mg of free base) per day via an oral route of administration. Another particularly effective amount is 120 mg of raloxifene hydrochloride per day via an oral route of administration. Such dosages will be administered to a patient in need of treatment from one to three times each day or as often as needed to effectively inhibit cataracts. Raloxifene hydrochloride may be administered for extended periods of time including six months to two years, specifically including about one year. Raloxifene hydrochloride may be used for repeated courses or continuously for an indefinite time.

The formulations which follow are given for purposes of illustration and are not intended to be limiting in any way. The total active ingredient in such formulations comprises from 0.1% to 99.9% by weight of the formulation. The term, “active ingredient” means a compound of formula I, or a pharmaceutical salt or solvate thereof, (preferably raloxifene hydrochloride). An even more preferred formulation of a compound of formula I would be raloxifene hydrochloride in the particular crystalline form, particle size, and composition illustrated in U.S. Pat. No. 5,731,327 and PCT application WO 97/35571 (Oct, 2, 1997) the teachings of each are incorporated by reference.

### Formulation 1

### Gelatin Capsules

| Ingredient | Quantity (mg/capsule) |
|---|---|
| Active Ingredient | 50-600 |
| Starch NF | 0-500 |
| Starch flowable powder | 0-500 |
| Silicone fluid 350 centistrokes | 0-15 |

The ingredients are blended, passed through a No. 45 mesh U.S. sieve, and filled into hard gelatin capsules.

### Formulation 2

### Tablets

| Ingredient | Quantity (mg/tablet) |
|---|---|
| Active Ingredient | 50-600 |
| Starch | 10-50 |
| Cellulose, microcrystalline | 10-20 |
| Polyvinylpyrrolidone | 5 |
| (as 10% solution in water) | |
| Sodium carboxymethyl cellulose | 5 |
| Magnesium stearate | 1 |
| Talc | 1-5 |

The active ingredient, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules thus produced are dried at 50-60° C. and passed through a No. 18 mesh U.S. sieve. The sodium carboxymethyl cellulose, magnesium stearate, and talc, previously passed through a No. 60 mesh U.S. sieve, are added to the above granules and thoroughly mixed. The resultant material is compressed in a tablet forming machine to yield the tablets.

### Formulation 3

### Aerosol

| Ingredient | Weight % |
|---|---|
| Active Ingredient | 0.50 |
| Ethanol | 29.50 |
| Propellant 22 | 70.00 |
| (Chlorodifluoromethane) | |

The active ingredient is mixed with ethanol and the mixture added to a portion of the propellant 22, cooled to −30° C. and transferred to a filling device. The required amount is then fed to a stainless steel container and diluted with the remainder of the propellant. The valve units are then fitted to the container.

### Formulation 4

### Suspension

| Ingredient | Weight/Volume | |
|---|---|---|
| Active Ingredient | 100 | mg |
| Sodium carboxymethyl | 50 | mg |
| cellulose | | |
| Syrup | 1.25 | mL |
| Benzoic acid solution (0.1 M) | 0.10 | mL |
| Flavor | q.v. | |
| Color | q.v. | |
| Purified water to total | 5 | mL |

Suspensions each containing 100 mg of a compound of formula I per 5 mL dose are prepared as follows: the active ingredient is passed through a No. 45 mesh U.S. sieve and mixed with the sodium carboxymethyl cellulose and syrup to form a smooth paste. The benzoic acid solution, flavor, and color diluted in water are added and mixture stirred thoroughly. Additional water is added to bring the entire mixture to the required volume.

### EXAMPLE

A phase 3, multicenter, double-blind, placebo-controlled, randomized clinical trial was conducted. The trial included 7705 women (mean age 67 years old), who were an average of 19 years postmenopausal. These patients were randomly assigned to the following treatment protocol: raloxifene hydrochloride at 60 mg per day via oral administration, raloxifene at 120 mg per day, or placebo. The study was conducted for a period of 48 months. As a portion of the patient's overall evaluation, various clinical tests and parameters were measured at periodic intervals. An analysis of adverse events relating to cataracts reported in this trial revealed a trend toward decreasing risk of cataracts with raloxifene, as compared to placebo. Furthermore, this trend appears to be dose dependent, with patients on the 120 mg per day dose experiencing fewer events than patients on the 60 mg per day dose, and patients on both doses experiencing fewer events than those on placebo.

The following tables provide summaries of select cataract treatment-emergent signs and symptoms (“TESS”) events for various patient populations at the thirty-six and the forty-eight month mark of the clinical trial.

TABLE 1
All Randomized Patients - 36-Month Data

| Event | | | | | |
|---|---|---|---|---|---|
| Classifi- | | | | | p- |
| cation | Placebo | Rlx60 | Rlx120 | Comparison | value |
| Any | (N = 2576) | (N = 2557) | (N = 2572) | Overall | 0.252 |
| Cata- | 124 (4.8%) | 105 (4.1%) | 101 (3.9%) | | |
| ract - | | | | Pla v. Rlx | 0.103 |
| related | | | | Pla v. 060 | 0.220 |
| event | | | | Pla v. 120 | 0.120 |
| | | | | 060 v. 120 | 0.743 |
| “Cata- | (N = 2576) | (N = 2557) | (N = 2572) | Overall | 0.115 |
| ract | 110 (4.3%) | 92 (3.6%) | 82 (3.2%) | | |
| Speci- | | | | Pla v. Rlx | 0.054 |
| fied” | | | | Pla v. 060 | 0.216 |
| | | | | Pla v. 120 | 0.041 |
| | | | | 060 v. 120 | 0.418 |
| Surgery | (N = 2576) | (N = 2557) | (N = 2572) | Overall | 0.899 |
| for | 56 (2.2%) | 60 (2.3%) | 60 (2.3%) | | |
| cataract | | | | Pla v. Rlx | 0.646 |
| | | | | Pla v. 060 | 0.677 |
| | | | | Pla v. 120 | 0.701 |
| | | | | 060 v. 120 | 0.974 |

TABLE 2
Patients Reporting a Cataract-Related Current
Condition at Baseline - 36-Month Data

| Event | | | | | |
|---|---|---|---|---|---|
| Classifi- | | | | | p- |
| cation | Placebo | Rlx60 | Rlx120 | Comparison | value |
| Any | (N = 170) | (N = 163) | (N = 154) | Overall | 0.450 |
| Cata- | 28 (16.5%) | 25 (15.3%) | 18 (11.7%) | | |
| ract - | | | | Pla v. Rlx | 0.386 |
| related | | | | Pla v. 060 | 0.778 |
| event | | | | Pla v. 120 | 0.218 |
| | | | | 060 v. 120 | 0.343 |

TABLE 3
Patients Not Reporting a Cataract-Related Current
Condition at Baseline - 36-Month Data

| Event | | | | | |
|---|---|---|---|---|---|
| Classifi- | | | | | p- |
| cation | Placebo | Rlx60 | Rlx120 | Comparison | value |
| Any | (N = 2406) | (N = 2394) | (N = 2418) | Overall | 0.425 |
| Cata- | 96 (4.0%) | 80 (3.3%) | 83 (3.4%) | | |
| ract | | | | Pla v. Rlx | 0.194 |
| related- | | | | Pla v. 060 | 0.232 |
| event | | | | Pla v. 120 | 0.306 |
| | | | | 060 v. 120 | 0.862 |

TABLE 4
All Randomized Patients - 48-Month Data

| Event | | | | | |
|---|---|---|---|---|---|
| Classifi- | | | | | p- |
| cation | Placebo | Rlx60 | Rlx120 | Comparison | value |
| Any | (N = 2576) | (N = 2557) | (N = 2572) | Overall | 0.498 |
| Cata- | 160 (6.2%) | 151 (5.9%) | 140 (5.4%) | | |
| ract | | | | Pla v. Rlx | 0.343 |
| related | | | | Pla v. 060 | 0.646 |
| event | | | | Pla v. 120 | 0.240 |
| | | | | 060 v. 120 | 0.474 |
| “Cata- | (N = 2576) | (N = 2557) | (N = 2572) | Overall | 0.472 |
| ract | 141 (5.5%) | 135 (5.3%) | 122 (4.7%) | | |
| Speci- | | | | Pla v. Rlx | 0.386 |
| fied” | | | | Pla v. 060 | 0.758 |
| | | | | Pla v. 120 | 0.234 |
| | | | | 060 v. 120 | 0.379 |
| Surgery | (N = 2576) | (N = 2557) | (N = 2572) | Overall | 0.783 |
| for | 86 (3.3%) | 85 (3.3%) | 78 (3.0%) | | |
| cataract | | | | Pla v. Rlx | 0.707 |
| | | | | Pla v. 060 | 0.977 |
| | | | | Pla v. 120 | 0.532 |
| | | | | 060 v. 120 | 0.552 |

TABLE 5
Patients Reporting a Cataract-Related Current
Condition at Baseline - 48-Month Data

| Event | | | | | |
|---|---|---|---|---|---|
| Classifi- | | | | | p- |
| cation | Placebo | Rlx60 | Rlx120 | Comparison | value |
| Any | (N = 176) | (N = 172) | (N = 164) | Overall | 0.670 |
| Cata- | 33 (18.8%) | 31 (18.0%) | 25 (15.2%) | | |
| ract- | | | | Pla v. Rlx | 0.555 |
| related | | | | Pla v. 060 | 0.861 |
| event | | | | Pla v. 120 | 0.390 |
| | | | | 060 v. 120 | 0.494 |

TABLE 6
Patients Not Reporting a Cataract-Related Current
Condition at Baseline - 48-Month Data

| Event | | | | | |
|---|---|---|---|---|---|
| Classifi- | | | | | p- |
| cation | Placebo | Rlx60 | Rlx120 | Comparison | value |
| Any | (N = 2400) | (N = 2385) | (N = 2408) | Overall | 0.716 |
| Cata- | 127 (5.3%) | 120 (5.0%) | 115 (4.8%) | | |
| ract- | | | | Pla v. Rlx | 0.477 |
| related | | | | Pla v. 060 | 0.684 |
| event | | | | Pla v. 120 | 0.413 |
| | | | | 060 v. 120 | 0.682 |

While the effect of estrogen on cataracts has not been completely described to date, investigators have noted in limited observational trials that use of oral estrogen in postmenopausal women is associated with a seeming reduction in the incidence of cataracts (Klein B E, Klein R, Ritter L L. Is there evidence of an estrogen effect on age-related lens opacities? The Beaver Dam Eye Study [see comments]. Arch Ophthal 112:85-91, 1994). Unfortunately, to date, the largest prospective clinical trial of oral estrogen (the HERS trial), was too small to see an effect of estrogen on cataracts (Hulley S, Grady D, Bush T, Furberg C, Herrington D, Riggs B, Vittinghoff. Randomized trial of estrogen plus progestin for secondary prevention of coronary heart disease in postmenopausal women. JAMA 280: 605-613, 1998). The mechanism for this seeming reduction in cataracts may be due to oral estrogen's ability to ameliorate the damaging effects of TGF-beta on the lens (Hales A M, Chamberlain C G, Murphy C R, McAvoy J W, Estrogen protects lenses against cataract induced by transforming growth factor-beta. J Exp Med 185:273-80, 1997.

With the exception of tamoxifen, the effect of SERMs on cataracts has not been reported. In the largest prospective clinical trial performed with tamoxifen to date (the NSABP's P-1 trial), tamoxifen was associated with a relative risk of cataracts of 1.14 with a borderline significant 95% CI of 1.01-1.29 (Fisher B, Costantino J P, Wickerham D L, Redmond C K, Kavanah M, Cronin WM, Vogel V, Robidoux A, Dimitrov N, Atkins J, Daly M, Wieand S, Tan-Chiu E, Ford L, Wolmark N. Tamoxifen for prevention of breast cancer: Report of the National Surgical Adjuvant Breast and Bowel Project P-1 Study. J Natl Cancer Inst 90: 1371-1388, 1998). Presumably, tamoxifen is acting as an anti-estrogen in the lens. Studies in animals have suggested that one possible mechanism by which tamoxifen increases the risk of cataracts may be by blocking chloride channels. Chloride channels in the lens are essential for maintaining normal lens hydration and transmittance. In organ culture, tamoxifen blocked chloride channels and led to lens opacity associated with cataracts at clinically relevant concentrations (Zhang J J, Jacob T J, Valverde M A, Hardy S P, Mintenig G M, Sepulveda F V, Gill D R, Hyde S C, Trezise A E, Higgins C F. Tamoxifen blocks chloride channels. A possible mechanism for cataract formation. J Clin Invest 94:1690-7, 1994).

It is reasonable to expect that tamoxifen and raloxifene, although they are both SERMs, have different effects on the lens, since they are of different chemical classes and have been shown to differ in their effects on at least one other organ system, namely, the uterus. Tamoxifen has been shown to cause endometrial stimulation and rarely, endometrial cancer Fisher B et al., supra. Raloxifene, on the other hand, is not associated with endometrial stimulation and has not been associated with endometrial cancer (Davies GC, Huster W J, Shen W, Mitlak B., Plouffe L, Shah A, Cohen F J. Endometrial response to raloxifene compared with placebo, cyclical hormone replacement therapy and unopposed estrogen in postmenopausal women. Menopause 6:188-195, 1999 and Goldstein S R, Scheele W H, Rajagopalan SK, Wilkie J, Walsh BW, Parsons AK. A 12-month comparative study of raloxifene, estrogen and placebo on the postmenopausal endometrium. Obstet Gynecol 95: 95-103, 2000 and Cohen FJ, Watts S, Shah A, Akers. R, Plouffe L. Uterine effects of 3-year raloxifene therapy in postmenopausal women younger than age 60. Obstet Gynecol 95:104-110, 2000).

We claim:

1. A method for inhibiting cataracts comprising administering to a patient in need thereof an effective amount of a compound of the formula 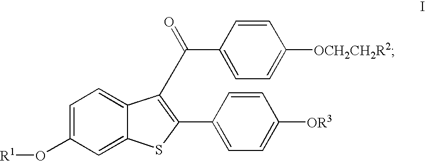
or a pharmaceutical salt or solvate thereof wherein:
R¹ and R³ are independently hydrogen, methyl, benzoyl, substituted benzoyl, or C(O)—(C₁-C₆ alkyl);
R² is selected from the group pyrolidin-l-yl, piperidin-1-yl, and hexamethyleneimin-1-yl; where the R² group is optionally the N-oxide.

2. A method according to claim 1 wherein said patient is a human

3. A method according to claim 2 wherein said human is a female.

4. A method according to claim 3 wherein said female is estrogen deficient.

5. A method according to claim 2 wherein said compound of formula I is a pharmaceutical acid addition salt, R¹ and R³ are hydrogen, and R² is piperidin-1-yl.

6. A method according to claim 5 wherein said compound of formula I is the hydrochloride salt.

7. A method according to claim 2 wherein said compound of formula I is a pharmaceutical acid addition salt, R¹ and R³ are hydrogen, and R² is pyrolidin-1-yl.

8. A method according to claim 7 wherein said compound of formula I is the hydrochloride salt.

9. A method according to claim 4 wherein said compound of formula I is a pharmaceutical acid addition salt, R¹ and R³ are hydrogen, and R² is piperidin-1-yl.

10. A method according to claim 9 wherein said compound of formula I is the hydrochloride salt.

11. A method according to claim 4 wherein said compound of formula I is a pharmaceutical acid addition salt, R¹ and R³ are hydrogen, and R² is pyrolidin-1-yl.

12. A method according to claim 11 wherein said compound of formula I is the hydrochloride salt.

13. The use of a compound of the formula 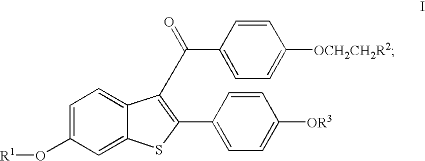
or a pharmaceutical salt or solvate thereof wherein:
R¹ and R³ are independently hydrogen, methyl, benzoyl, substituted benzoyl, or C(O)—(C₁-C₆ alkyl);
R² is selected from the group pyrolidin-1-yl, piperidin-1-yl, and hexamethyleneimin-1-yl; where the R² group is optionally the N-oxide, in the preparation of a medicament for inhibiting cataracts.

14. A use according to claim 13 wherein said medicament is adapted for human use.

15. A use according to claim 14 wherein said human is a female.

16. A use according to claim 15 wherein said female is estrogen deficient.

17. A use according to claims 13 to 16 wherein said compound of formula I is a pharmaceutical acid addition salt, R¹ and R³ are hydrogen, and R² is piperidin-1-yl.

18. A use according to claims 13 to 17 wherein said compound of formula I is the hydrochloride salt.

19. A use according to claims 13 to 16 wherein said compound of formula I is a pharmaceutical acid addition salt, R¹ and R³ are hydrogen, and R² is pyrolidin-1-yl.

20. A use according to claim 19 wherein said compound of formula I is the hydrochloride salt.

21. A compound of the formula 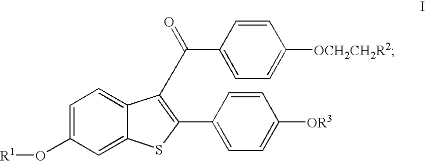
or a pharmaceutical salt or solvate thereof wherein:
R¹ and R³ are independently hydrogen, methyl, benzoyl, substituted benzoyl, or C(O)-(C₁-C₆ alkyl);
R² is selected from the group pyrolidin-1-yl, piperidin-1-yl, and hexamethyleneimin-1-yl; where the R² group is optionally the N-oxide; for use in inhibiting cataracts.

22. A compound according to claim 21 wherein said compound of formula I is a pharmaceutical acid addition salt, R¹ and R³ are hydrogen, and R² is piperidin-1-yl.

23. A compound according to claim 21 wherein said compound of formula I is the hydrochloride salt.

24. A compound according to claim 21 wherein said compound of formula I is a pharmaceutical acid addition salt, R¹ and R³ are hydrogen, and R² is pyrolidin-1-yl.

25. A compound according to claim 21 wherein said compound of formula I is the hydrochloride salt.
